SEQUENCE LISTING (13 sequences)

SEQ ID NO: 1 - DNA, 40 bp, Homo sapiens
caggaaccagccaagtgcccccttcaagggacggcaggag

SEQ ID NO: 2 - DNA, 40 bp, Homo sapiens
gagtttcactggtgtgtcagacactggaagaattgtctta

SEQ ID NO: 3 - DNA, 81 bp, Homo sapiens
ccttcaagggacggcaggaggaggggtgttctggaaacgtggactgctggccaagccccctgagtttcactggtgtgtcag

SEQ ID NO: 4 - DNA, 2556 bp, Homo sapiens
atggggtggctttgctctgggctcctgttccctgtgagctgcctggtcctgctgcaggtggcaagctctgggaacatgaaggtcttgcaggagcccacctgcgtctccgactacatgagcatctctacttgcgagtggaagatgaatggtcccaccaattgcagcaccgagctccgcctgttgtaccagctggtttttctgctctccgaagcccacacgtgtatccctgagaacaacggaggcgcggggtgcgtgtgccacctgctcatggatgacgtggtcagtgcggataactatacactggacctgtgggctgggcagcagctgctgtggaagggctccttcaagcccagcgagcatgtgaaacccagggccccaggaaacctgacagttcacaccaatgtctccgacactctgctgctgacctggagcaacccgtatccccctgacaattacctgtataatcatctcacctatgcagtcaacatttggagtgaaaacgacccggcagatttcagaatctataacgtgacctacctagaaccctccctccgcatcgcagccagcaccctgaagtctgggatttcctacagggcacgggtgagggcctgggctcagtgctataacaccacctggagtgagtggagccccagcaccaagtggcacaactcctacagggagcccttcgagcagcacctcctgctgggcgtcagcgtttcctgcattgtcatcctggccgtctgcctgttgtgctatgtcagcatcaccaagattaagaaagaatggtgggatcagattcccaacccagcccgcagccgcctcgtggctataataatccaggatgctcaggggtcacagtgggagaagcggtcccgaggccaggaaccagccaagtgcccccttcaagggacggcaggaggaggggtgttctggaaacgtggactgctggccaagccccctgagtttcactggtgtgtcagacactggaagaattgtcttaccaagctcttgccctgttttctggagcacaacatgaaaagggatgaagatcctcacaaggctgccaaagagatgcctttccagggctctggaaaatcagcatggtgcccagtggagatcagcaagacagtcctctggccagagagcatcagcgtggtgcgatgtgtggagttgtttgaggccccggtggagtgtgaggaggaggaggaggtagaggaagaaaaagggagcttctgtgcatcgcctgagagcagcagggatgacttccaggagggaagggagggcattgtggcccggctaacagagagcctgttcctggacctgctcggagaggagaatgggggcttttgccagcaggacatgggggagtcatgccttcttccaccttcgggaagtacgagtgctcacatgccctgggatgagttcccaagtgcagggcccaaggaggcacctccctggggcaaggagcagcctctccacctggagccaagtcctcctgccagcccgacccagagtccagacaacctgacttgcacagagacgcccctcgtcatcgcaggcaaccctgcttaccgcagcttcagcaactccctgagccagtcaccgtgtcccagagagctgggtccagacccactgctggccagacacctggaggaagtagaacccgagatgccctgtgtcccccagctctctgagccaaccactgtgccccaacctgagccagaaacctgggagcagatcctccgccgaaatgtcctccagcatggggcagctgcagcccccgtctcggcccccaccagtggctatcaggagtttgtacatgcggtggagcagggtggcacccaggccagtgcggtggtgggcttgggtcccccaggagaggctggttacaaggccttctcaagcctgcttgccagcagtgctgtgtccccagagaaatgtgggtttggggctagcagtggggaagaggggtataagcctttccaagacctcattcctggctgccctggggaccctgccccagtccctgtccccttgttcacctttggactggacagggagccacctcgcagtccgcagagctcacatctcccaagcagctccccagagcacctgggtctggagccgggggaaaaggtagaggacatgccaaagcccccacttccccaggagcaggccacagacccccttgtggacagcctgggcagtggcattgtctactcagcccttacctgccacctgtgcggccacctgaaacagtgtcatggccaggaggatggtggccagacccctgtcatggccagtccttgctgtggctgctgctgtggagacaggtcctcgccccctacaacccccctgagggccccagacccctctccaggtggggttccactggaggccagtctgtgtccggcctccctggcaccctcgggcatctcagagaagagtaaatcctcatcatccttccatcctgcccctggcaatgctcagagctcaagccagacccccaaaatcgtgaactttgtctccgtgggacccacatacatgagggtctct

SEQ ID NO: 5 - protein, 852 aa, Homo sapiens
MGWLCSGLLFPVSCLVLLQVASSGNMKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQHLLLGVSVSCIVILAVCLLCYVSITKIKKEWWDQIPNPARSRLVAIIIQDAQGSQWEKRSRGQEPAKCPLQGTAGGGVFWKRGLLAKPPEFHWCVRHWKNCLTKLLPCFLEHNMKRDEDPHKAAKEMPFQGSGKSAWCPVEISKTVLWPESISVVRCVELFEAPVECEEEEEVEEEKGSFCASPESSRDDFQEGREGIVARLTESLFLDLLGEENGGFCQQDMGESCLLPPSGSTSAHMPWDEFPSAGPKEAPPWGKEQPLHLEPSPPASPTQSPDNLTCTETPLVIAGNPAYRSFSNSLSQSPCPRELGPDPLLARHLEEVEPEMPCVPQLSEPTTVPQPEPETWEQILRRNVLQHGAAAAPVSAPTSGYQEFVHAVEQGGTQASAVVGLGPPGEAGYKAFSSLLASSAVSPEKCGFGASSGEEGYKPFQDLIPGCPGDPAPVPVPLFTFGLDREPPRSPQSSHLPSSSPEHLGLEPGEKVEDMPKPPLPQEQATDPLVDSLGSGIVYSALTCHLCGHLKQCHGQEDGGQTPVMASPCCGCCCGDRSSPPTTPLRAPDPSPGGVPLEASLCPASLAPSGISEKSKSSSSFHPAPGNAQSSSQTPKIVNFVSVGPTYMRVS

SEQ ID NO: 6 - DNA, 20 bp, Artificial Sequence
  synthetic oligonucleotide probe
ccaagtgcccccttcaaggg

SEQ ID NO: 7 - DNA, 20 bp, Artificial Sequence
  synthetic oligonucleotide probe
ggtgtgtcagacactggaag

SEQ ID NO: 8 - protein, 10 aa, Homo sapiens
PAKCPLQGTA

SEQ ID NO: 9 - protein, 10 aa, Homo sapiens
HWCVRHWKNC

SEQ ID NO: 10 - DNA, 27 bp, Artificial Sequence
  synthetic oligonucleotide
gtctgcctgttgtgctatgtcagcatc

SEQ ID NO: 11 - DNA, 28 bp, Artificial Sequence
  synthetic oligonucleotide
ccagaggactgtcttgctgatctccact

SEQ ID NO: 12 - DNA, 19 bp, Artificial Sequence
  synthetic oligonucleotide
atggggtggctttgctctg

SEQ ID NO: 13 - DNA, 23 bp, Artificial Sequence
  synthetic oligonucleotide
agagaccctcatgtatgtgggtc

(19) United States
(12) Patent Application Publication
(10) Pub. No.: US 2005/0260650 A1
(43) Pub. Date: Nov. 24, 2005

(54) Alternatively spliced isoforms of interleukin-4 receptor subunit alpha (IL-4Ralpha)

(76) Inventors: John C. Castle, Seattle, WA (US); Philip W. Garrett-Engele, Seattle, WA (US); Zhengyan Kan, Bellevue, WA (US); Christopher D. Armour, Kirkland, WA (US); Christopher K. Raymond, Seattle, WA (US)

Correspondence Address: R. Douglas Bradley;Merck & Co., Inc., P.O. Box 2000 - Patent Department RY60-30, Rahway, NJ, 07065-0907, US

(21) Appl. No.: 11/104,923

(22) Filed: Apr. 12, 2005

Related U.S. Application Data
(60) Provisional application No. 60/561,828, filed on Apr. 13, 2004.
(60) Provisional application No. 60/564,261, filed on Apr. 21, 2004.

(51) Int. Cl. C12Q 1/68; G01N 33/53; G01N 33/567; C07H 21/04; C12P 21/04; C07K 14/54

(52) U.S. Cl. 435/6; 435/7.2; 435/69.52; 435/320.1; 435/325; 530/351; 536/23.5

(57) ABSTRACT

The present invention features nucleic acids and polypeptides encoding a novel splice variant isoform of interleukin 4, subunit alpha (IL-4Rα). The polynucleotide sequence of IL-4Rαsv1 is provided by SEQ ID NO 4. The amino acid sequence for IL-4Rαsv1 is provided by SEQ ID NO 5. The present invention also provides methods for using IL-4Rαsv1 polynucleotides and proteins to screen for compounds that bind to IL-4Rαsv1.

This application claims priority to U.S. Provisional Patent Application Ser. No. 60/561,828 filed on Apr. 13, 2004, and U.S. Provisional Patent Application Ser. No. 60/564,261 filed on Apr. 21, 2004, each of which is incorporated by reference herein in its entirety.

### BACKGROUND OF THE INVENTION

The references cited herein are not admitted to be prior art to the claimed invention.

The cytokines interleukin-4 (IL-4), also known as B-cell stimulating factor (BSF-1), and interleukin-13 (IL-13) are produced by CD4⁺ T helper type 2 (T_{H}2) cells, and basophils and mast cells, in response to receptor-mediated events (Nelms et al., 1999 Annu. Rev. Immunol., 17:701-738). T_{H}2 differentiation of antigen-stimulated naïve T-cells, IgE production, chemokine and mucus production, and eosinophil recruitment and activation characterize the inflammatory response induced by T_{H}2 cytokines, such as IL-4 (reviewed in Pernis and Rothman, 2002 J. Clin. Invest., 109:1279-1283). Other effects of IL-4 on hematopoietic cells include: increased expression of class II MHC molecules in B cells (Noelle et al., 1984 Proc. Natl. Acad. Sci. USA, 81:6149-6153), upregulation of IL-4 receptor expression (Ohara and Paul, 1988 Proc. Natl. Acad. Sci. USA, 85:6107-6011), and promotion of B cell growth (Howard et al., 1982 J. Exp. Med., 155:914-923). IL-4 can also prolong the lives of B and T lymphocytes in culture (Hu-Li et al., 1987, J. Exp. Med., 165:157-172). While a strong humoral response, driven by T_{H}2-type cytokines, is useful in parasite clearance, it may also be involved in allergy, asthma, and autoimmunity when directed against an innocuous antigen.

The biological activities of IL-4 are mediated by specific receptors. IL-4 receptors are composed of two transmembrane proteins. IL-4 interacts with an IL-4Rα subunit with high affinity, leading to dimerization of the complex with another protein. If IL-4Rα interacts with the common gamma chain (γC) (also a component of IL-2, -7, -9, -15, and -21 receptors) on hematopoietic cells, then a type I receptor is generated. In non-hematopoietic cells, dimerization of IL-4Rα with IL-13Rα1 forms the type II receptor. IL-13 binds with moderate affinity to IL-13Rα1, the complex of which recruits IL-4Rα to form the type II receptor (reviewed in Kelly-Welch et al., 2003 Science, 300:1527-1528). Both subunits of the IL-4 receptor mediate cellular activation, but only the IL-4Rα subunit is required for initial IL-4 binding (Yin et al., 1994 J. Biol. Chem. 269:26614-26617).

Additionally, a soluble form of IL-4Rα (sIL-4Rα), which lacks the transmembrane and intracellular regions but retains the extracellular ligand binding portion, has been detected in humans, suggesting a possible immunoregulatory role for IL-4 activity (Jung et al., 1999 Int. Arch. Allergy Immunol., 119:23-30). Unlike the murine form of sIL-4Rα, human sIL-4Rα does not appear to result from alternative splicing, but rather from cleavage by metalloproteinases (Mosley et al., 1989 Cell, 59:335-348; Idzerda et al., 1990 J. Exp. Med., 171:861-873; Jung et al., 1999 Int. Arch. Allergy Immunol., 119:23-30).

The cytoplasmic portions of IL-4Rα and IL-13R subunits interact with the Janus family of receptor-associated kinases (JAK). IL-4Rα associates with JAK1 and γC with JAK3, while IL-13Rα1 interacts with JAK2 or TYK2. Dimerization of IL-4R stimulates JAK, which phosphorylates tyrosine residues in the cytoplasmic region of the IL-4Rα chain. Signaling molecules which contain Src homology 2 (SH2) domains or protein tyrosine binding domains (PTBs) can then dock at the phosphorylated receptor. One of the key signaling pathways activated by IL-4R involves signal transducer and activator of transcription-6 (STAT6), a latent cytoplasmic transcription factor. Upon binding the phosphorylated receptor through its SH2 domain, STAT6 also undergoes tyrosine phosphorylation. The phosphorylated STAT6 disengages from IL-4Rα, homodimerizes, and translocates to the nucleus where it binds to the consensus sequences within promoters of IL-4 and IL-13 regulated genes, such as those involved in allergic responses. STAT6 also regulates genes involved in lymphocyte growth and survival (reviewed in Kelly-Welch et al., 2003 Science, 300:1527-1528; Nelms et al., 1999 Annu. Rev. Immunol., 17:701-738).

IL-4 and IL-4R have also been implicated in non-immune functions, such as muscle growth. NFATc2 isoform (nuclear factor of activated T-cells transcription factor) has been reported to regulate the expression of IL-4 in muscle cells. IL-4 is expressed by a subset of muscle cells undergoing myoblast fusion (nascent myotube), and interacts with the IL-4Rα subunits on myoblasts to promote fusion with the myotube and muscle growth. Muscle cells in IL-4^{-/-} or IL-4Rα^{-/-}mice form normally, but are reduced in size and in myonuclear number (Horsley et al., 2003 Cell, 133:483-494).

The human IL-4Rα mRNA transcript (NM_{—}000418) consists of 11 exons, of which the latter 9 exons encode 825 amino acids. Exons 1 and 2 of the IL-4Rα transcript (NM_{—}000418) represent untranslated portions of the IL-4Rα mRNA. The IL-4Rα protein is composed of a signal sequence (amino acids 1-25), an external domain (amino acids 26-231), a transmembrane domain (amino acids 232-255), and a large cytoplasmic domain (amino acids 256-825) (Galizzi et al., 1990 Int. Immunol., 2:669-675). IL-4Rα is a member of the hematopoietin receptor superfamily, which has distinct features, such as, four conserved cysteine residues and a WSXWS motif in the extracellular region (Idzerda et al., 1990 J. Exp. Med. 171:861-873). The human IL-4Rα protein has 52% homology to mouse IL-4Rα protein, and 50% homology to the rat IL-4Rα protein. IL-4Rα is expressed primarily in B- and T-cells, hematopoietic, endothelial, epithelial, muscle, fibroblast, and hepatocyte cells, and brain tissues, suggesting a broad range of action for the IL-4 cytokine (Ohara and Paul, 1987 Nature, 325:537-540; Lowenthal et al, 1988 J. Immunol., 140:456-464). A number of human tumor cell lines have also been found to over-express IL-4Rα, such as renal cell carincoma, squamous cell carcinoma of the head and neck, malignant glioma, lung tumor, and breast cancer (reviewed in Kawakami et al., 2001 Crit. Rev. Immunol., 21:299-310).

Variations in IL-4Rα may result in altered IL-4 responsiveness. An Ile50Val substitution in IL-4Rα has been shown to be associated with atopic asthma. The Ile50Val variant also demonstrates enhanced signaling, resulting in increased STAT6 activation and IgE production (Mitsuyasu et al., 1998 Nat. Gen. 19: 199-120; Mitsuyasu et al., 1999 J. Immunol. 1227-1231). An Arg576Gln mutation in IL-4Rα, associated with hyper-IgE syndrome and atopic dermatitis, induced enhanced signaling function, specifically, higher levels of low affinity IgE receptors (CD23) expression on peripheral blood mononuclear cells (Hershey et al., 1997 N. Engl. J. Med. 337:1720-1725). Additonally, the Arg576Gln mutation in IL-4Rα decreases binding of SHP-1 molecules to an adjacent phosphorylated tyrosine at position 575. SHP-1 dephosphorylates regulatory phophotyrosines and has been implicated in signaling termination of cytokine receptors (Yi et al, 1993 Mol. Cell. Biol. 13:7577-7586; Klingmuller et al., 1995 Cell 80:729-738; Chen et al., 1996 Mol. Cell. Biol. 16:3685-3697). Decreased binding of SHP-1 to phophorylated Y575 in IL-4Rα may result in enhanced receptor signaling. Schulte et al. (1997 J. Exp. Med. 186:1419-1429) describes allelic variations in mouse IL-4Rα which are associated with altered ligand binding. IL-4Rα polymorphisms also have been found to associate with other immune-related diseases, such as type I diabetes (Bugawan et al., 2003 Am. J. Hum. Genet., 72:1505-14; Mirel et al., 2002 Diabetes, 51:3336-3341).

IL-4Rα mediated signaling pathways are believed to play a crucial role in allergic diseases. Studies with IL-4 deficient mice show that it is important for allergy-induced IgE production, airway hyperresponsiveness, and eosinophilia (Kips et al., 1995 Int. Arch. Allergy. Immunol. 107:115-8; Coyle et al. 1995 Am. J. Respir. Cell. Mol. Biol. 13:54-59; Hogan et al., 1997 J. Clin. Invest. 99:1329-1339). Using knockout mice, Cohn et al. (1999 J. Immunol. 162:6178-6183) demonstrated that signaling through IL-4Rα is critical for T_{H}2-induced airway mucus production. IL-4Rα^{-/-} mice are also unable to induce IgE production upon allergen sensitization (Grunewald et al., 1998 J. Immunol. 160:4004-4009).

IL-4Rα signaling may also influence allograft rejection. The Q576R IL-4Rα variant is also associated with decreased kidney allograft survival (Hackenstein et al., 1999 Tissue Antigens 54:471-7). Fanslow et al. (1991 J. Immunol. 147:535-40) demonstrated that treatment with recombinant IL-4Rα increased survival of allografts in mice.

Targeting of the IL-4Rα signaling pathways has been the subject of interest for the treatment of atopic disorders, such as asthma or allergic rhinitis (reviewed in Jarnicki and Fallon, 2003, Curr. Opin. Pharmacol. 3:449-455; Barnes, 2001, J. Allergy Clin. Immunol. 108: S72-S76). IL-4Rα signaling pathways can be modified by several compounds and proteins. The immunosuppressive drug leflunomide, which inhibits tyrosine kinase activity, blocks IL-4-mediated tyrosine phosphorylation of JAK3 and STAT6 and decreases binding of STAT6 to DNA in B-cells (Siemasko et al., 1998 J. Immunol. 160:1581-1588). Corticosteroids also interfere with IL-4Rα signaling through inhibition of STAT6 phosphorylation and DNA binding (So et al., FEBS Lett. 2002 518:53-59). Aspirin and salicylates also inhibit activation of STAT6 by IL-4 (Perez-G. et al., 2002 J. Immunol. 168:1428-1434). An endogenous protein, suppressor of cytokine signaling protein 1 (SOCS-1), is a potent inhibitor of IL-4Rα signaling, blocking the activation of JAK1 and STAT6 (Losman et al., 1999 J. Immunol. 162:3770-3774). A synthetic peptide, corresponding to an IL-4Rα cytoplasmic domain critical for signal transduction, can diminish IL-4 induced proliferation of responsive cells (Izuhara et al., 1995 Cell. Immunol. 163:254-259). Antagonistic IL-4 mutants have also been identified which are able to bind the IL-4Rα subunit, but do not induce IL-4Rα signaling functions (Grunewald et al., 1997 J. Biol. Chem. 272:1480-1483; Grunewald et al., 1998 J. Immunol. 160:4004-4009; Schnare et al., 1998 J. Immunol. 161:3484-3492).

Because of the multiple therapeutic values of drugs targeting the IL-4Rα signaling pathway, there is a need in the art for compounds that selectively bind to isoforms of IL-4Rα. The present invention is directed towards a novel IL-4Rα isoform (IL-4Rαsv1) and uses thereof.

### BRIEF DESCRIPTION OF THE FIGURES

FIG. 1A illustrates the exon structure of IL-4Rα mRNA corresponding to the known long reference form of IL-4Rα mRNA (labeled NM_{—}000418) and the exon structure corresponding to the inventive splice variant (labeled IL-4Rαsv1). FIG. 1B depicts the nucleotide sequences of the exon junctions resulting from the splicing of exon 10 to intron 10A (SEQ ID NO 1) and intron 10A to exon 11 in the case of IL-4Rαsv1 mRNA (SEQ ID NO 2), where intron 10A consists of a portion of intron 10 and is encoded by 5′ CCTTCAAGGGACGG CAGGAGGAGGGGTGTTCTGGAAACGTGGACTGCTGGCCAAGCCCCCTGAGTTTCAC TGGTGTGTCAG 3′ (SEQ ID NO 3). In FIG. 1B, SEQ ID NO 1, the nucleotides shown in italics represent the 20 nucleotides at the 3′ end of exon 10 and the nucleotides shown in underline represent the 20 nucleotides at the 5′ end of intron 10A; in the case of SEQ ID NO 2, the nucleotides shown in italics represent the 20 nucleotides at the 3′ end of intron 10A and the nucleotides shown in underline represent the 20 nucleotides at the 5′ end of exon 11.

### SUMMARY OF THE INVENTION

Microarray experiments and RT-PCR have been used to identify and confirm the presence of novel splice variants of human IL-4Rα mRNA. More specifically, the present invention features polynucleotides encoding different protein isoforms of IL-4Rα. A polynucleotide sequence encoding IL-4Rαsv1 is provided by SEQ ID NO 4. An amino acid sequence for IL-4Rαsv1 is provided by SEQ ID NO 5.

Thus, a first aspect of the present invention describes a purified IL-4Rαsv1 encoding nucleic acid. The IL-4Rαsv1 encoding nucleic acid comprises SEQ ID NO 4 or the complement thereof. Reference to the presence of one region does not indicate that another region is not present. For example, in different embodiments the inventive nucleic acid can comprise, consist, or consist essentially of an encoding nucleic acid sequence of SEQ ID NO 4.

Another aspect of the present invention describes a purified IL-4Rαsv1 polypeptide that can comprise, consist or consist essentially of the amino acid sequence of SEQ ID NO 5.

Another aspect of the present invention describes expression vectors. In one embodiment of the invention, the inventive expression vector comprises a nucleotide sequence encoding a polypeptide comprising, consisting, or consisting essentially of SEQ ID NO 5, wherein the nucleotide sequence is transcriptionally coupled to an exogenous promoter.

Alternatively, the nucleotide sequence comprises, consists, or consists essentially of SEQ ID NO 4, and is transcriptionally coupled to an exogenous promoter.

Another aspect of the present invention describes recombinant cells comprising expression vectors comprising, consisting, or consisting essentially of the above-described sequences and the promoter is recognized by an RNA polymerase present in the cell. Another aspect of the present invention describes a recombinant cell made by a process comprising the step of introducing into the cell an expression vector comprising a nucleotide sequence comprising, consisting, or consisting essentially of SEQ ID NO 4, or a nucleotide sequence encoding a polypeptide comprising, consisting, or consisting essentially of an amino acid sequence of SEQ ID NO 5, wherein the nucleotide sequence is transcriptionally coupled to an exogenous promoter. The expression vector can be used to insert recombinant nucleic acid into the host genome or can exist as an autonomous piece of nucleic acid.

Another aspect of the present invention describes a method of producing IL-4Rαsv1 polypeptide comprising SEQ ID NO 5. The method involves the step of growing a recombinant cell containing an inventive expression vector under conditions wherein the polypeptide is expressed from the expression vector.

Another aspect of the present invention features a purified antibody preparation comprising an antibody that binds selectively to IL-4Rαsv1 as compared to one or more IL-4Rα isoform polypeptides that are not IL-4Rsv1α.

Another aspect of the present invention provides a method of screening for a compound that binds to IL-4Rαsv1, or fragments thereof. In one embodiment, the method comprises the steps of: (a) expressing a polypeptide comprising the amino acid sequence of SEQ ID NO 5 or a fragment thereof from recombinant nucleic acid; (b) providing to said polypeptide a labeled IL-4Rα ligand that binds to said polypeptide and a test preparation comprising one or more test compounds; (c) and measuring the effect of said test preparation on binding of said test preparation to said polypeptide comprising SEQ ID NO 5.

In another embodiment of the method, a compound is identified that binds selectively to IL-4Rαsv1 polypeptide as compared to one or more IL-4Rα isoform polypeptides that are not IL-4Rαsv1. This method comprises the steps of: providing a IL-4Rαsv1 polypeptide comprising SEQ ID NO 5; providing a IL-4Rα isoform polypeptide that is not IL-4Rαsv1; contacting said IL-4Rαsv1 polypeptide and said IL-4Rα isoform polypeptide that is not IL-4Rαsv1 with a test preparation comprising one or more test compounds; and determining the binding of said test preparation to said IL-4Rαsv1 polypeptide and to IL-4Rα isoform polypeptide that is not IL-4Rαsv1, wherein a test preparation that binds to said IL-4Rαsv1 polypeptide but does not bind to said IL-4Rα isoform polypeptide that is not IL-4Rαsv1 contains a compound that selectively binds said IL-4Rαsv1 polypeptide.

In another embodiment of the invention, a method is provided for screening for a compound able to bind to or interact with a IL-4Rαsv1 protein or a fragment thereof comprising the steps of: expressing a IL-4Rαsv1 polypeptide comprising SEQ ID NO 5 or a fragment thereof from a recombinant nucleic acid; providing to said polypeptide a labeled IL-4Rα ligand that binds to said polypeptide and a test preparation comprising one or more compounds; and measuring the effect of said test preparation on binding of said labeled IL-4Rα ligand to said polypeptide, wherein a test preparation that alters the binding of said labeled IL-4Rα ligand to said polypeptide contains a compound that binds to or interacts with said polypeptide.

Other features and advantages of the present invention are apparent from the additional descriptions provided herein, including the different examples. The provided examples illustrate different components and methodology useful in practicing the present invention. The examples do not limit the claimed invention. Based on the present disclosure the skilled artisan can identify and employ other components and methodology useful for practicing the present invention.

### DEFINITIONS

Unless defined otherwise, all technical and scientific terms used herein have the meaning commonly understood by one of ordinary skill in the art to which this invention belongs.

As used herein, “IL-4Rα” refers to human interleukin 4 receptor subunit α (IL-4Rα) protein (NP_{—}000409), also known as CD 124. In contrast, reference to an IL-4Rα isoform includes NP_{—}000409 and other polypeptide isoform variants of IL-4Rα.

As used herein, “IL-4Rαsv1” refers to a splice variant isoform of human IL-4Rα protein, wherein the splice variants have the amino acid sequence set forth in SEQ ID NO 5 (for IL-4Rαsv1).

As used herein, “IL-4Rα” refers to polynucleotides encoding IL-4Rα.

As used herein, “IL-4Rαsv1” refers to polynucleotides encoding IL-4Rαsv1 having an amino acid sequence set forth in SEQ ID NO 5.

As used herein, an “isolated nucleic acid” is a nucleic acid molecule that exists in a physical form that is nonidentical to any nucleic acid molecule of identical sequence as found in nature; “isolated” does not require, although it does not prohibit, that the nucleic acid so described has itself been physically removed from its native environment. For example, a nucleic acid can be said to be “isolated” when it includes nucleotides and/or internucleoside bonds not found in nature. When instead composed of natural nucleosides in phosphodiester linkage, a nucleic acid can be said to be “isolated” when it exists at a purity not found in nature, where purity can be adjudged with respect to the presence of nucleic acids of other sequence, with respect to the presence of proteins, with respect to the presence of lipids, or with respect to the presence of any other component of a biological cell, or when the nucleic acid lacks sequence that flanks an otherwise identical sequence in an organism's genome, or when the nucleic acid possesses sequence not identically present in nature. As so defined, “isolated nucleic acid” includes nucleic acids integrated into a host cell chromosome at a heterologous site, recombinant fusions of a native fragment to a heterologous sequence, recombinant vectors present as episomes or as integrated into a host cell chromosome.

A “purified nucleic acid” represents at least 10% of the total nucleic acid present in a sample or preparation. In preferred embodiments, the purified nucleic acid represents at least about 50%, at least about 75%, or at least about 95% of the total nucleic acid in a isolated nucleic acid sample or preparation. Reference to “purified nucleic acid” does not require that the nucleic acid has undergone any purification and may include, for example, chemically synthesized nucleic acid that has not been purified.

The phrases “isolated protein”, “isolated polypeptide”, “isolated peptide” and “isolated oligopeptide” refer to a protein (or respectively to a polypeptide, peptide, or oligopeptide) that is nonidentical to any protein molecule of identical amino acid sequence as found in nature; “isolated” does not require, although it does not prohibit, that the protein so described has itself been physically removed from its native environment. For example, a protein can be said to be “isolated” when it includes amino acid analogues or derivatives not found in nature, or includes linkages other than standard peptide bonds. When instead composed entirely of natural amino acids linked by peptide bonds, a protein can be said to be “isolated” when it exists at a purity not found in nature—where purity can be adjudged with respect to the presence of proteins of other sequence, with respect to the presence of non-protein compounds, such as nucleic acids, lipids, or other components of a biological cell, or when it exists in a composition not found in nature, such as in a host cell that does not naturally express that protein.

As used herein, a “purified polypeptide” (equally, a purified protein, peptide, or oligopeptide) represents at least 10% of the total protein present in a sample or preparation, as measured on a weight basis with respect to total protein in a composition. In preferred embodiments, the purified polypeptide represents at least about 50%, at least about 75%, or at least about 95% of the total protein in a sample or preparation. A “substantially purified protein” (equally, a substantially purified polypeptide, peptide, or oligopeptide) is an isolated protein, as above described, present at a concentration of at least 70%, as measured on a weight basis with respect to total protein in a composition. Reference to “purified polypeptide” does not require that the polypeptide has undergone any purification and may include, for example, chemically synthesized polypeptide that has not been purified.

As used herein, the term “antibody” refers to a polypeptide, at least a portion of which is encoded by at least one immunoglobulin gene, or fragment thereof, and that can bind specifically to a desired target molecule. The term includes naturally-occurring forms, as well as fragments and derivatives. Fragments within the scope of the term “antibody” include those produced by digestion with various proteases, those produced by chemical cleavage and/or chemical dissociation, and those produced recombinantly, so long as the fragment remains capable of specific binding to a target molecule. Among such fragments are Fab, Fab′, Fv, F(ab)′₂, and single chain Fv(scFv) fragments. Derivatives within the scope of the term include antibodies (or fragments thereof) that have been modified in sequence, but remain capable of specific binding to a target molecule, including: interspecies chimeric and humanized antibodies; antibody fusions; heteromeric antibody complexes and antibody fusions, such as diabodies (bispecific antibodies), single-chain diabodies, and intrabodies (see, e.g., Marasco (ed.), Intracellular Antibodies: Research and Disease Applications, Springer-Verlag New York, Inc. (1998) (ISBN: 3540641513). As used herein, antibodies can be produced by any known technique, including harvest from cell culture of native B lymphocytes, harvest from culture of hybridomas, recombinant expression systems, and phage display.

As used herein, a “purified antibody preparation” is a preparation where at least 10% of the antibodies present bind to the target ligand. In preferred embodiments, antibodies binding to the target ligand represent at least about 50%, at least about 75%, or at least about 95% of the total antibodies present. Reference to “purified antibody preparation” does not require that the antibodies in the preparation have undergone any purification.

As used herein, “specific binding” refers to the ability of two molecular species concurrently present in a heterogeneous (inhomogeneous) sample to bind to one another in preference to binding to other molecular species in the sample. Typically, a specific binding interaction will discriminate over adventitious binding interactions in the reaction by at least two-fold, more typically by at least 10-fold, often at least 100-fold; when used to detect analyte, specific binding is sufficiently discriminatory when determinative of the presence of the analyte in a heterogeneous (inhomogeneous) sample. Typically, the affinity or avidity of a specific binding reaction is least about 1 μM.

The term “antisense”, as used herein, refers to a nucleic acid molecule sufficiently complementary in sequence, and sufficiently long in that complementary sequence, as to hybridize under intracellular conditions to (i) a target mRNA transcript or (ii) the genomic DNA strand complementary to that transcribed to produce the target mRNA transcript.

The term “subject”, as used herein refers to an organism and to cells or tissues derived therefrom. For example the organism may be an animal, including but not limited to animals such as cows, pigs, horses, chickens, cats, dogs, etc., and is usually a mammal, and most commonly human.

### DETAILED DESCRIPTION OF THE INVENTION

This section presents a detailed description of the present invention and its applications. This description is by way of several exemplary illustrations, in increasing detail and specificity, of the general methods of this invention. These examples are non-limiting, and related variants that will be apparent to one of skill in the art are intended to be encompassed by the appended claims.

The present invention relates to the nucleic acid sequences encoding human IL-4Rαsv1 that is an alternatively spliced isoform of IL-4Rα, and to the amino acid sequences encoding this proteins. SEQ ID NO 4 is a polynucleotide sequence representing an exemplary open reading frame that encodes the IL-4Rαsv1 protein. SEQ ID NO 5 shows the polypeptide sequence of IL-4Rαsv1.

IL-4Rαsv1 polynucleotide sequence encoding IL-4Rαsv1 protein, as exemplified and enabled herein include a number of specific, substantial and credible utilities. For example, IL-4Rαsv1 encoding nucleic acid was identified in an mRNA sample obtained from a human source (see Example 1). Such nucleic acids can be used as hybridization probes to distinguish between cells that produce IL-4Rαsv1 transcripts from human or non-human cells (including bacteria) that do not produce such transcripts. Similarly, antibodies specific for IL-4Rαsv1 can be used to distinguish between cells that express IL-4Rαsv1 from human or non-human cells (including bacteria) that do not express IL-4Rαsv1.

IL-4Rα is an important drug target for the management of immune function and T_{H}2 cytokine-induced inflammation responses, as well as diseases such as asthma, allergic rhinitis, allergic dermatitis, allograft rejection and cancer (Borish et al., 2001 J. Allergy Clin. Immunol. 107:963-970; Wright et al., 1999 Laryngoscope 109:551-556; Nasert et al., 1995 Behring Inst Mitt. 96:118-30; Fanslow et al., 1991 J. Immunol. 147:535-40; Kawakami et al., 2001 Crit. Rev. Immunol. 21:299-310). Given the potential importance of IL-4Rα activity to the therapeutic management of a wide array of diseases, it is of value to identify IL-4Rα isoforms and identify IL-4Rα-ligand compounds that are isoform specific, as well as compounds that are effective ligands for two or more different IL-4Rα isoforms. In particular, it may be important to identify compounds that are effective inhibitors of a specific IL-4Rα isoform activity, yet do not bind to or interact with a plurality of different IL-4Rα isoforms. Compounds that bind to or interact with multiple IL-4Rα isoforms may require higher drug doses to saturate multiple IL-4Rα-isoform binding sites and thereby result in a greater likelihood of secondary non-therapeutic side effects. Furthermore, biological effects could also be caused by the interaction of a drug with the IL-4Rαsv1 isoform specifically. For the foregoing reasons, IL-4Rαsv1 protein represents a useful compound binding target and has utility in the identification of new IL-4Rα-ligands exhibiting a preferred specificity profile and having greater efficacy for their intended use.

In some embodiments, IL-4Rαsv1 activity is modulated by a ligand compound to achieve one or more of the following: prevent or reduce the risk of occurrence, or recurrence of diseases resulting from allergic responses, such as asthma, allergic dermatitis, and allergic rhinitis, and allograft rejection.

Compounds modulating IL-4Rαsv1 include agonists, antagonists, and allosteric modulators. While not wishing to be limited to any particular theory of therapeutic efficacy, generally, but not always, IL-4Rαsv1 compounds will be used to modulate the IL-4Rα signaling activity. Compounds may interfere with binding of IL-4 to cell surface receptors or interfere with signal transduction; the site of action may be extracellular or intracellular. Therefore, agents that modulate IL-4Rα activity may be used to achieve a therapeutic benefit for any disease or condition due to, or exacerbated by, abnormal levels of IL-4Rα protein or its activity.

IL-4Rαsv1 activity can also be affected by modulating the cellular abundance of transcripts encoding IL-4Rαsv1. Compounds modulating the abundance of transcripts encoding IL-4Rαsv1 include a cloned polynucleotide encoding IL-4Rαsv1 that can express IL-4Rαsv1 in vivo, antisense nucleic acids targeted to IL-4Rαsv1 transcripts, and enzymatic nucleic acids, such as ribozymes and RNAi, targeted to IL-4Rαsv1 transcripts.

In some embodiments, IL-4Rαsv1 is modulated to achieve a therapeutic effect upon diseases in which regulation of IL-4Rα is desirable. For example, allergies and asthma may be treated by modulating IL-4Rαsv1 activity. In other embodiments, cancer may be treated by targeting IL-4Rαsv1 expressed on tumors.

IL-4Rαsv1 Nucleic Acids

IL-4Rαsv1 nucleic acids contain regions that encode for polypeptides comprising, consisting, or consisting essentially of SEQ ID NO 5. The IL-4Rαsv1 nucleic acid has a variety of uses, such as use as a hybridization probe or PCR primer to identify the presence of IL-4Rαsv1; use as a hybridization probe or PCR primer to identify nucleic acids encoding for proteins related to IL-4Rαsv1; and/or use for recombinant expression of IL-4Rαsv1 polypeptides. In particular, IL-4Rαsv1 polynucleotides have an additional polynucleotide region that consists of intron 10A (SEQ ID NO 3) of the IL-4Rα gene.

Regions in IL-4Rαsv1 nucleic acid that do not encode for IL-4Rαsv1, or are not found in SEQ ID NO 4, if present, are preferably chosen to achieve a particular purpose. Examples of additional regions that can be used to achieve a particular purpose include: a stop codon that is effective at protein synthesis termination; capture regions that can be used as part of an ELISA sandwich assay; reporter regions that can be probed to indicate the presence of the nucleic acid; expression vector regions; and regions encoding for other polypeptides.

The guidance provided in the present application can be used to obtain the nucleic acid sequence encoding IL-4Rαsv1 related proteins from different sources. Obtaining nucleic acids IL-4Rαsv1 related proteins from different sources is facilitated by using sets of degenerative probes and primers and the proper selection of hybridization conditions. Sets of degenerative probes and primers are produced taking into account the degeneracy of the genetic code. Adjusting hybridization conditions is useful for controlling probe or primer specificity to allow for hybridization to nucleic acids having similar sequences.

Techniques employed for hybridization detection and PCR cloning are well known in the art. Nucleic acid detection techniques are described, for example, in Sambrook, et al., in Molecular Cloning, A Laboratory Manual, 2^{nd} Edition, Cold Spring Harbor Laboratory Press, 1989. PCR cloning techniques are described, for example, in White, Methods in Molecular Cloning, volume 67, Humana Press, 1997.

IL-4Rαsv1 probes and primers can be used to screen nucleic acid libraries containing, for example, cDNA. Such libraries are commercially available, and can be produced using techniques such as those described in Ausubel, Current Protocols in Molecular Biology, John Wiley, 1987-1998.

Starting with a particular amino acid sequence and the known degeneracy of the genetic code, a large number of different encoding nucleic acid sequences can be obtained. The degeneracy of the genetic code arises because almost all amino acids are encoded for by different combinations of nucleotide triplets or “codons”. The translation of a particular codon into a particular amino acid is well known in the art (see, e.g., Lewin GENES IV, p. 119, Oxford University Press, 1990). Amino acids are encoded for by codons as follows:
A═Ala═Alanine: codons GCA, GCC, GCG, GCU
C═Cys═Cysteine: codons UGC, UGU
D═Asp═Aspartic acid: codons GAC, GAU
E═Glu═Glutamic acid: codons GAA, GAG
F═Phe═Phenylalanine: codons UUC, UUU
G═Gly═Glycine: codons GGA, GGC, GGG, GGU
H═His═Histidine: codons CAC, CAU
I═Ile═Isoleucine: codons AUA, AUC, AUU
K═Lys═Lysine: codons AAA, AAG
L═Leu═Leucine: codons UUA, UUG, CUA, CUC, CUG, CUU
M═Met═Methionine: codon AUG
N═Asn═Asparagine: codons AAC, AAU
P═Pro═Proline: codons CCA, CCC, CCG, CCU
Q═Gln═Glutamine: codons CAA, CAG
R═Arg═Arginine: codons AGA, AGG, CGA, CGC, CGG, CGU
S═Ser═Serine: codons AGC, AGU, UCA, UCC, UCG, UCU
T═Thr═Threonine: codons ACA, ACC, ACG, ACU
V═Val═Valine: codons GUA, GUC, GUG, GUU
W═Trp═Tryptophan: codon UGG
Y═Tyr═Tyrosine: codons UAC, UAU

Nucleic acid having a desired sequence can be synthesized using chemical and biochemical techniques. Examples of chemical techniques are described in Ausubel, Current Protocols in Molecular Biology, John Wiley, 1987-1998, and Sambrook et al., in Molecular Cloning, A Laboratory Manual, 2^{nd} Edition, Cold Spring Harbor Laboratory Press, 1989. In addition, long polynucleotides of a specified nucleotide sequence can be ordered from commercial vendors, such as Blue Heron Biotechnology, Inc. (Bothell, Wash.).

Biochemical synthesis techniques involve the use of a nucleic acid template and appropriate enzymes such as DNA and/or RNA polymerases. Examples of such techniques include in vitro amplification techniques such as PCR and transcription based amplification, and in vivo nucleic acid replication. Examples of suitable techniques are provided by Ausubel, Current Protocols in Molecular Biology, John Wiley, 1987-1998, Sambrook et al., in Molecular Cloning, A Laboratory Manual, 2^{nd} Edition, Cold Spring Harbor Laboratory Press, 1989, and U.S. Pat. No. 5,480,784.

IL-4Rαsv1 Probes

Probes for IL-4Rαsv1 contain a region that can specifically hybridize to IL-4Rαsv1 target nucleic acids under appropriate hybridization conditions and can distinguish IL-4Rαsv1 nucleic acids from non-target nucleic acids, in particular IL-4Rα polynucleotides not containing intron 10A. Probes for IL-4Rαsv1 can also contain nucleic acid regions that are not complementary to IL-4Rαsv1 nucleic acids.

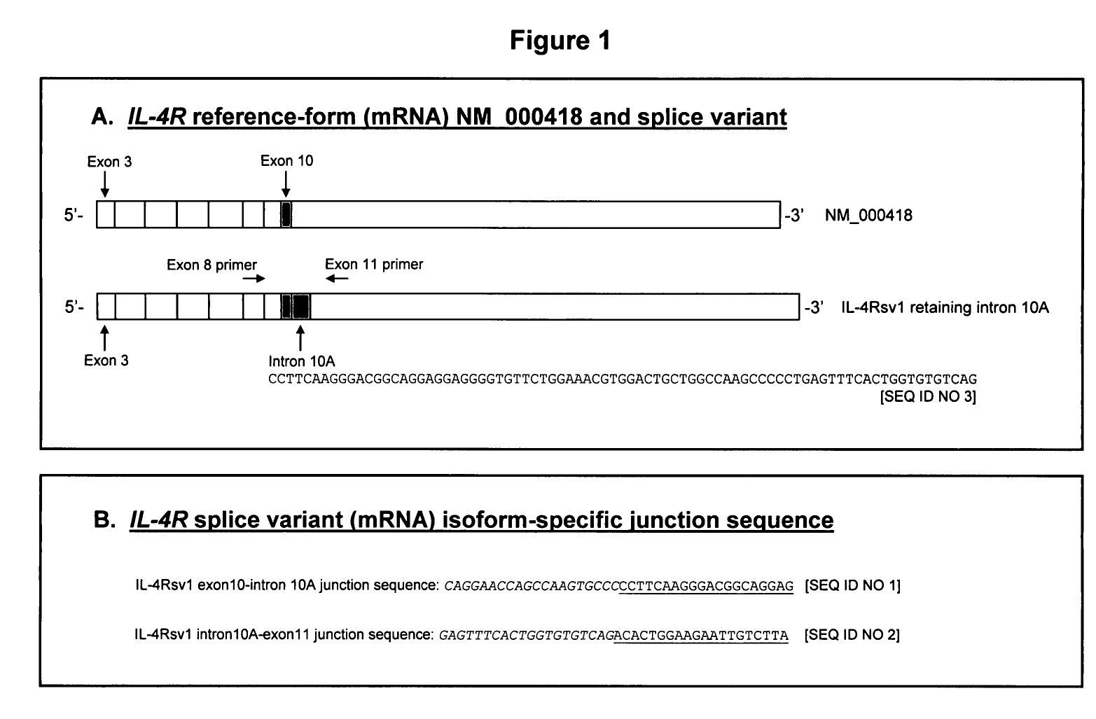

In embodiments where, for example, IL-4Rαsv1 polynucleotide probes are used in hybridization assays to specifically detect the presence of IL-4Rαsv1 polynucleotides in samples, the IL-4Rαsv1 polynucleotides comprise at least 20 nucleotides of the IL-4Rαsv1 sequence that correspond to the novel exon junction polynucleotide regions. In particular, for detection of IL-4Rαsv1, the probe comprises at least 20 nucleotides of the IL-4Rαsv1 sequence that corresponds to an exon junction polynucleotide created by the alternative splicing of exon 10 to intron 10A of the primary transcript of the IL-4Rα gene (see FIGS. 1A and 1B). For example, the polynucleotide sequence: 5′ CCAAGTGCCCCCTTCAAGGG 3′ [SEQ ID NO 6] represents one embodiment of such an inventive IL-4Rαsv1 polynucleotide wherein a first 10 nucleotides region is complementary and hybridizable to the 3′ end of exon 10 of the IL-4Rα gene and a second 10 nucleotides region is complementary and hybridizable to the 5′ end of intron 10A of the IL-4Rα gene (see FIG. 1B). In another example, the polynucleotide sequence: 5′ GGTG TGTCAGACACTGGAAG 3′ [SEQ ID NO 7] represents one embodiment of such an inventive IL-4Rαsv1 polynucleotide wherein a first 10 nucleotides region is complementary and hybridizable to the 3′ end of intron 10A of the IL-4Rα gene and a second 10 nucleotides region is complementary and hybridizable to the 5′ end of exon 11 of the IL-4Rα gene (see FIG. 1B).

In some embodiments, the first 20 nucleotides of an IL-4Rαsv1 probe comprise a first continuous region of 5 to 15 nucleotides that is complementary and hybridizable to the 3′ end of exon 10 and a second continuous region of 5 to 15 nucleotides that is complementary and hybridizable to the 5′ end of intron 10A of the IL-4Rα gene, or alternatively the first 20 nucleotides of an IL-4Rαsv1 probe comprise a first continous region of 5 to 15 nucleotides that is complementary and hybridizable to the 3′ end of intron 10A and a second continuous region of 5 to 15 nucleotides that is complementary and hybridizable to the 5′ end of exon 11.

In other embodiments, the IL-4Rαsv1 polynucleotide comprises at least 40, 60, 80 or 100 nucleotides of the IL-4Rαsv1 sequence that correspond to a junction polynucleotide region created by the lack of splicing of exon 10 to exon 11 resulting in the retention of intron 10A of the primary transcript of the IL-4Rα gene. In embodiments involving IL-4Rαsv1, the IL-4Rαsv1 polynucleotide is selected to comprise a first continuous region of at least 5 to 15 nucleotides that is complementary and hybridizable to the 3′ end of exon 10 and a second continuous region of at least 5 to 15 nucleotides that is complementary and hybridizable to the 5′ end of intron 10A, or the IL-4Rαsv1 polynucleotide is selected to comprise a first continuous region of at least 5 to 15 nucleotides that is complementary and hybridizable to the 3′ end of intron 10A and a second continuous region of at least 5 to 15 nucleotides that is complementary and hybridizable to the 5′ end of exon 11 of the IL-4Rα gene. As will be apparent to a person of skill in the art, a large number of different polynucleotide sequences from the region of the exon 10 to intron 10A or intron 10A to exon 11 splice junctions may be selected which will, under appropriate hybridization conditions, have the capacity to detectably hybridize to IL-4Rαsv1 polynucleotides, and yet will hybridize to a much less extent or not at all to IL-4Rα isoform polynucleotides wherein exon 10 is not spliced to intron 10A or wherein intron 10A is not spliced to exon 11.

Preferably, non-complementary nucleic acid that is present has a particular purpose such as being a reporter sequence or being a capture sequence. However, additional nucleic acid need not have a particular purpose as long as the additional nucleic acid does not prevent the IL-4Rαsv1 nucleic acid from distinguishing between target polynucleotides, e.g., IL-4Rαsv1 polynucleotides, and non-target polynucleotides, including, but not limited to IL-4Rα polynucleotides not comprising the exon 10 to intron 10A or intron 10A to exon 11 splice junctions found in IL-4Rαsv1.

Hybridization occurs through complementary nucleotide bases. Hybridization conditions determine whether two molecules, or regions, have sufficiently strong interactions with each other to form a stable hybrid.

The degree of interaction between two molecules that hybridize together is reflected by the melting temperature (Tₘ) of the produced hybrid. The higher the Tₘ the stronger the interactions and the more stable the hybrid. Tₘ is effected by different factors well known in the art such as the degree of complementarity, the type of complementary bases present (e.g., A-T hybridization versus G-C hybridization), the presence of modified nucleic acid, and solution components (e.g., Sambrook, et al., in Molecular Cloning, A Laboratory Manual, 2^{nd} Edition, Cold Spring Harbor Laboratory Press, 1989).

Stable hybrids are formed when the Tₘ of a hybrid is greater than the temperature employed under a particular set of hybridization assay conditions. The degree of specificity of a probe can be varied by adjusting the hybridization stringency conditions. Detecting probe hybridization is facilitated through the use of a detectable label. Examples of detectable labels include luminescent, enzymatic, and radioactive labels.

Examples of stringency conditions are provided in Sambrook, et al., in Molecular Cloning, A Laboratory Manual, 2^{nd} Edition, Cold Spring Harbor Laboratory Press, 1989. An example of high stringency conditions is as follows: Prehybridization of filters containing DNA is carried out for 2 hours to overnight at 65° C. in buffer composed of 6×SSC, 5× Denhardt's solution, and 100 μg/ml denatured salmon sperm DNA. Filters are hybridized for 12 to 48 hours at 65° C. in prehybridization mixture containing 100 μg/ml denatured salmon sperm DNA and 5-20×10⁶ cpm of ³²P-labeled probe. Filter washing is done at 37° C. for 1 hour in a solution containing 2×SSC, 0.1% SDS. This is followed by a wash in 0.1×SSC, 0.1% SDS at 50° C. for 45 minutes before autoradiography. Other procedures using conditions of high stringency would include, for example, either a hybridization step carried out in 5×SSC, 5× Denhardt's solution, 50% formamide at 42° C. for 12 to 48 hours or a washing step carried out in 0.2×SSPE, 0.2% SDS at 65° C. for 30 to 60 minutes.

Recombinant Expression

IL-4Rαsv1 polynucleotides, such as those comprising SEQ ID NO 4, can be used to make IL-4Rαsv1. In particular, IL-4Rsv1 can be expressed from recombinant nucleic acids in a suitable host or in vitro using a translation system. Recombinantly expressed IL-4Rαsv1 polypeptides can be used, for example, in assays to screen for compounds that bind IL-4Rαsv1. Alternatively, IL-4Rαsv1 polypeptides can also be used to screen for compounds that bind to one or more IL-4Rα isoforms, but do not bind to IL-4Rαsv1.

In some embodiments, expression is achieved in a host cell using an expression vector. An expression vector contains recombinant nucleic acid encoding a polypeptide along with regulatory elements for proper transcription and processing. The regulatory elements that may be present include those naturally associated with the recombinant nucleic acid and exogenous regulatory elements not naturally associated with the recombinant nucleic acid. Exogenous regulatory elements such as an exogenous promoter can be useful for expressing recombinant nucleic acid in a particular host.

Generally, the regulatory elements that are present in an expression vector include a transcriptional promoter, a ribosome binding site, a terminator, and an optionally present operator. Another preferred element is a polyadenylation signal providing for processing in eukaryotic cells. Preferably, an expression vector also contains an origin of replication for autonomous replication in a host cell, a selectable marker, a limited number of useful restriction enzyme sites, and a potential for high copy number. Examples of expression vectors are cloning vectors, modified cloning vectors, and specifically designed plasmids and viruses.

Expression vectors providing suitable levels of polypeptide expression in different hosts are well known in the art. Mammalian expression vectors well known in the art include, but are not restricted to, pcDNA3 (Invitrogen, Carlsbad Calif.), pSecTag2 (Invitrogen), pMC1neo (Stratagene, La Jolla Calif.), pXT1 (Stratagene), pSG5 (Stratagene), pCMVLacl (Stratagene), pCI-neo (Promega), EBO-pSV2-neo (ATCC 37593), pBPV-1(8-2) (ATCC 37110), pdBPV-MMTneo(342-12) (ATCC 37224), pRSVgpt (ATCC 37199), pRSVneo (ATCC 37198), pSV2-dhfr (ATCC 37146) and pUCTag (ATCC 37460). Bacterial expression vectors well known in the art include pET11a (Novagen), pBluescript SK (Stratagene, La Jolla), pQE-9 (Qiagen Inc., Valencia), lambda gt11 (Invitrogen), pcDNAII (Invitrogen), and pKK223-3 (Pharmacia). Fungal cell expression vectors well known in the art include pPICZ (Invitrogen), pYES2 (Invitrogen), and Pichia expression vector (Invitrogen). Insect cell expression vectors well known in the art include Blue Bac III (Invitrogen), pBacPAK8 (CLONTECH, Inc., Palo Alto) and PfastBacHT (Invitrogen, Carlsbad).

Recombinant host cells may be prokaryotic or eukaryotic. Examples of recombinant host cells include the following: bacteria such as E. coli; fungal cells such as yeast; mammalian cells such as human, bovine, porcine, monkey and rodent; and insect cells such as Drosophila and silkworm derived cell lines. Commercially available mammalian cell lines include L cells L-M(TK⁻) (ATCC CCL 1.3), L cells L-M (ATCC CCL 1.2), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C127I (ATCC CRL 1616), BS-C-1 (ATCC CCL 26) MRC-5 (ATCC CCL 171), and HEK 293 cells (ATCC CRL-1573).

To enhance expression in a particular host it may be useful to modify the sequence provided in SEQ ID NO 4 to take into account codon usage of the host. Codon usages of different organisms are well known in the art (see, Ausubel, Current Protocols in Molecular Biology, John Wiley, 1987-1998, Supplement 33 Appendix 1C).

Expression vectors may be introduced into host cells using standard techniques. Examples of such techniques include transformation, transfection, lipofection, protoplast fusion, and electroporation.

Nucleic acids encoding for a polypeptide can be expressed in a cell without the use of an expression vector employing, for example, synthetic mRNA or native mRNA. Additionally, mRNA can be translated in various cell-free systems such as wheat germ extracts and reticulocyte extracts, as well as in cell based systems, such as frog oocytes. Introduction of mRNA into cell based systems can be achieved, for example, by microinjection or electroporation.

IL-4Rαsv1 Polypeptides

IL-4Rαsv1 polypeptides contain an amino acid sequence comprising, consisting or consisting essentially of SEQ ID NO 5. IL-4Rαsv1 polypeptides have a variety of uses, such as providing a marker for the presence of IL-4Rαsv1; use as an immunogen to produce antibodies binding to IL-4Rαsv1; use as a target to identify compounds binding selectively to IL-4Rαsv1; or use in an assay to identify compounds that bind to one or more isoforms of IL-4Rα but do not bind to or interact with IL-4Rαsv1.

In chimeric polypeptides containing one or more regions from IL-4Rαsv1 and one or more regions not from IL-4Rαsv1, the region(s) not from IL-4Rαsv1, can be used, for example, to achieve a particular purpose or to produce a polypeptide that can substitute for IL-4Rαsv1, or fragments thereof. Particular purposes that can be achieved using chimeric IL-4Rαsv1 polypeptides include providing a marker for IL-4Rαsv1 activity, enhancing an immune response, and modulating the levels of IL-4Rα in the cell membrane or the activity of IL-4Rα.

Polypeptides can be produced using standard techniques including those involving chemical synthesis and those involving biochemical synthesis. Techniques for chemical synthesis of polypeptides are well known in the art (see e.g., Vincent, in Peptide and Protein Drug Delivery, New York, N.Y., Dekker, 1990).

Biochemical synthesis techniques for polypeptides are also well known in the art. Such techniques employ a nucleic acid template for polypeptide synthesis. The genetic code providing the sequences of nucleic acid triplets coding for particular amino acids is well known in the art (see, e.g., Lewin GENES IV, p. 119, Oxford University Press, 1990). Examples of techniques for introducing nucleic acid into a cell and expressing the nucleic acid to produce protein are provided in references such as Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Functional IL-4Rαsv1

Functional IL-4Rαsv1 is a different protein isoform of IL-4Rα. The identification of the amino acid and nucleic acid sequences of IL-4Rαsv1 provide tools for obtaining functional proteins related to IL-4Rαsv1 from other sources, for producing IL-4Rαsv1 chimeric proteins, and for producing functional derivatives of SEQ ID NO 5.

IL-4Rαsv1 polypeptides can be readily identified and obtained based on their sequence similarity to IL-4Rαsv1 (SEQ ID NO 5). In particular, IL 4Rαsv1 contains additional amino acids, encoded by nucleotides located after the splice junction that results from the retention of intron 10A (SEQ ID NO 3) of the IL-4Rα gene. The addition of intron 10A does not disrupt the protein reading frame as compared to the IL-4Rα reference sequence (NP_{—}000409). Therefore, IL-4Rαsv1 polypeptide contains 27 additional amino acids encoded by nucleotides corresponding to intron 10A (SEQ ID NO 3) of the IL-4Rα hnRNA as compared to the IL-4Rα reference sequence (NP_{—}000409).

Both the amino acid and nucleic acid sequences of IL-4Rαsv1 can be used to help identify and obtain IL-4Rαsv1. For example, SEQ ID NO 4 can be used to produce degenerative nucleic acid probes or primers for identifying and cloning nucleic acid polynucleotides encoding for an IL-4Rαsv1 polypeptide. In addition, polynucleotides comprising, consisting, or consisting essentially of SEQ ID NO 4 or fragments thereof, can be used under conditions of moderate stringency to identify and clone nucleic acids encoding IL-4Rαsv1 polypeptides from a variety of different organisms.

The use of degenerative probes and moderate stringency conditions for cloning is well known in the art. Examples of such techniques are described by Ausubel, Current Protocols in Molecular Biology, John Wiley, 1987-1998, and Sambrook, et al., in Molecular Cloning, A Laboratory Manual, 2^{nd} Edition, Cold Spring Harbor Laboratory Press, 1989.

Starting with IL-4Rαsv1 obtained from a particular source, derivatives can be produced. Such derivatives include polypeptides with amino acid substitutions, additions and deletions. Changes to IL-4Rαsv1 to produce a derivative having essentially the same properties should be made in a manner not altering the tertiary structure of IL-4Rαsv1.

Differences in naturally occurring amino acids are due to different R groups. An R group affects different properties of the amino acid such as physical size, charge, and hydrophobicity. Amino acids are can be divided into different groups as follows: neutral and hydrophobic (alanine, valine, leucine, isoleucine, proline, tryptophan, phenylalanine, and methionine); neutral and polar (glycine, serine, threonine, tryosine, cysteine, asparagine, and glutamine); basic (lysine, arginine, and histidine); and acidic (aspartic acid and glutamic acid).

Generally, in substituting different amino acids it is preferable to exchange amino acids having similar properties. Substituting different amino acids within a particular group, such as substituting valine for leucine, arginine for lysine, and asparagine for glutamine are good candidates for not causing a change in polypeptide functioning.

Changes outside of different amino acid groups can also be made. Preferably, such changes are made taking into account the position of the amino acid to be substituted in the polypeptide. For example, arginine can substitute more freely for nonpolar amino acids in the interior of a polypeptide then glutamate because of its long aliphatic side chain (See, Ausubel, Current Protocols in Molecular Biology, John Wiley, 1987-1998, Supplement 33 Appendix 1C).

IL-4Rαsv1 Antibodies

Antibodies recognizing IL-4Rαsv1 can be produced using a polypeptide containing SEQ ID NO 5 or a fragment thereof as an immunogen. Preferably, an IL-4Rαsv1 polypeptide used as an immunogen consists of a polypeptide of SEQ ID NO 5 or a SEQ ID NO 5 fragment having at least 10 contiguous amino acids in length corresponding to the polynucleotide region representing the junction resulting from the splicing of exon 10 to intron 10A or the junction resulting from the splicing of intron 10A to exon 11 of the IL-4Rα gene.

In some embodiments where, for example, IL-4Rαsv1 polypeptides are used to develop antibodies that bind specifically to IL-4Rαsv1 and not to other isoforms of IL-4Rα, the IL-4Rαsv1 polypeptides comprise at least 10 amino acids of the IL-4Rαsv1 polypeptide sequence corresponding to a junction polynucleotide region created by the retention of intron 10A of the primary transcript of the IL-4Rα gene (see FIG. 1). For example, the amino acid sequence: amino terminus-PAKCPLQGTA-carboxy terminus [SEQ ID NO 8] represents one embodiment of such an inventive IL-4Rαsv1 polypeptide wherein a first region of 4 amino acids is encoded by nucleotide sequence at the 3′ end of exon 10 of the IL-4Rα gene, the 5^{th} amino acid is encoded by the nucleotide sequence “CCC” at the exon junction of exon 10 and intron 10A, and a second region of 5 amino acids is encoded by the nucleotide sequence at the 5′ end of intron 10A. Preferably, at least 10 amino acids of the IL-4Rαsv1 polypeptide comprise a first continuous region of 2 to 8 amino acids that is encoded by nucleotides at the 3′ end of exon 10 and a second continuous region of 2 to 8 amino acids that is encoded by nucleotides at the 5′ end of intron 10A. The amino acid sequence: amino terminus-HWCVRHWKNC-carboxy terminus [SEQ ID NO 9] represents one embodiment of an inventive IL-4Rαsv1 polypeptide wherein a first 4 amino acid region is encoded by nucleotide sequence at the 3′ end of intron 10A of the IL-4Rα gene, the 5^{th} amino acid is encoded by the nucleotide sequence “AGA” at the exon junction of intron 10A and exon 11, and a second 5 amino acid region is encoded by the nucleotide sequence at the 5′ end of exon 11. Preferably, at least 10 amino acids of the IL-4Rαsv1 polypeptide comprise a first continuous region of 2 to 8 amino acids that is encoded by nucleotides at the 3′ end of intron 10A and a second continuous region of 2 to 8 amino acids that is encoded by nucleotides at the 5′ end of exon 11.

In other embodiments, IL-4Rαsv1-specific antibodies are made using an IL-4Rαsv1 polypeptide that comprises at least 20, 30, 40 or 50 amino acids of the IL-4Rαsv1 sequence that corresponds to a junction polynucleotide region created by the retention of intron 10A of the primary transcript of the IL-4Rα gene. In one case the IL-4Rαsv1 polypeptides are selected to comprise a first continuous region of at least 5 to 15 amino acids that is encoded by nucleotides at the 3′ end of exon 10 and a second continuous region of 5 to 15 amino acids that is encoded by nucleotides directly after the novel splice junction. Alternatively, IL-4Rαsv1 polypeptides are selected to comprise a first continuous region of at least 5 to 15 amino acids that is encoded by nucleotides at the 3′ end of intron 10A and a second continuous region of 5 to 15 amino acids that is encoded by nucleotides directly after the novel splice junction created by splicing of intron 10A to exon 11 of the IL-4Rα gene.

Antibodies to IL-4Rαsv1 have different uses, such as to identify the presence of IL-4Rαsv1 and to isolate IL-4Rαsv1 polypeptides. Identifying the presence of IL-4Rαsv1 can be used, for example, to identify cells producing IL-4Rαsv1. Such identification provides an additional source of IL-4Rαsv1 and can be used to distinguish cells known to produce IL-4Rαsv1 from cells that do not produce IL-4Rαsv1. For example, antibodies to IL-4Rαsv1 can distinguish human cells expressing IL-4Rαsv1 from human cells not expressing IL-4Rαsv1 or non-human cells (including bacteria) that do not express IL-4Rαsv1. Such IL-4Rαsv1 antibodies can also be used to determine the effectiveness of IL-4Rαsv1 ligands, using techniques well known in the art, to detect and quantify changes in the protein levels of IL-4Rαsv1 in cellular extracts, and in situ immunostaining of cells and tissues.

Techniques for producing and using antibodies are well known in the art. Examples of such techniques are described in Ausubel, Current Protocols in Molecular Biology, John Wiley, 1987-1998; Harlow, et al., Antibodies, A Laboratory Manual, Cold Spring Harbor Laboratory, 1988; and Kohler, et al., 1975 Nature 256:495-7.

IL-4Rαsv1 Binding Assay

A number of compounds known to modulate IL-4Rα signaling activity have been disclosed, including corticosteroids, leflunomide, and salicylates (So et al., 2002 FEBS Lett. 518:53-59; Siemasko et al., 1998 160:1581-1588; Perez-G et al., 2002 J. Immunol. 168:1428-1434). IL-4Rα induced activation of JAK1 and STAT6 is inhibited by SOCS-1 (Losman et al., 1999 J. Immunol. 162:3770-3774). Peptide sequences corresponding to a critical intracellular signaling region in IL-4Rα have also been reported to interfere with IL-4 induced proliferation (Izuhara et al., 1995 Cell. Immunol. 163:254-259). Cytokine traps consisting of the extracellular domains of the IL-4Rα and γ subunits fused to the Fc portionof human IgG1 have been shown to block IL-4 mediated T_{H}2 responses in mice in vivo (Economides et al., 2003 Nat. Med. 9:47-52). Antagonistic IL-4 mutants have also been described which inhibit IL-4 dependent responses (Tony et al., 1994 Eur. J. Biochem. 225:659-665; U.S. Pat. No. 6,028,176). Methods for screening IL-4 antagonists for their effects on IL-4Rα activity have been disclosed (see for example US 2003/0124121). Methods to show suppression of IL-4 mediated signaling in response to soluble IL-4Rα have also been provided in U.S. Pat. No. 5,840,869. A person skilled in the art may use these methods to screen IL-4Rαsv1 polypeptides for compounds that bind to, and in some cases functionally alter, each IL-4Rα isoform protein.

IL-4Rαsv1, or fragments thereof, can be used in binding studies to identify compounds binding to or interacting with IL-4Rαsv1, or fragments thereof. In one embodiment, IL-4Rαsv1, or a fragment thereof, can be used in binding studies with IL-4Rα isoform protein, or a fragment thereof, to identify compounds that: bind to or interact with IL-4Rαsv1 and other IL-4Rα isoforms; bind to or interact with one or more other IL-4Rα isoforms and not with IL-4Rαsv1. Such binding studies can be performed using different formats including competitive and non-competitive formats. Further competition studies can be carried out using additional compounds determined to bind to IL-4Rαsv1 or other IL-4Rα isoforms.

The particular IL-4Rαsv1 sequence involved in ligand binding can be identified using labeled compounds that bind to the protein and different protein fragments. Different strategies can be employed to select fragments to be tested to narrow down the binding region. Examples of such strategies include testing consecutive fragments about 15 amino acids in length starting at the N-terminus, and testing longer length fragments. If longer length fragments are tested, a fragment binding to a compound can be subdivided to further locate the binding region. Fragments used for binding studies can be generated using recombinant nucleic acid techniques.

In some embodiments, binding studies are performed using IL-4Rαsv1 expressed from a recombinant nucleic acid. Alternatively, recombinantly expressed IL-4Rαsv1 consists of the SEQ ID NO 5 amino acid sequence.

Binding assays can be performed using individual compounds or preparations containing different numbers of compounds. A preparation containing different numbers of compounds having the ability to bind to IL-4Rαsv1 can be divided into smaller groups of compounds that can be tested to identify the compound(s) binding to IL-4Rαsv1.

Binding assays can be performed using recombinantly produced IL-4Rαsv1 present in different environments. Such environments include, for example, cell extracts and purified cell extracts containing an IL-4Rαsv1 recombinant nucleic acid; and also include, for example, the use of a purified IL-4Rαsv1 polypeptide produced by recombinant means which is introduced into different environments.

In one embodiment of the invention, a binding method is provided for screening for a compound able to bind selectively to IL-4Rαsv1. The method comprises the steps: providing a IL-4Rαsv1 polypeptide comprising SEQ ID NO 5; providing a IL-4Rα isoform polypeptide that is not IL-4Rαsv1; contacting the IL-4Rαsv1 polypeptide and the IL-4Rα isoform polypeptide that is not IL-4Rαsv1 with a test preparation comprising one or more test compounds; and then determining the binding of the test preparation to the IL-4Rαsv1 polypeptide and to the IL-4Rα isoform polypeptide that is not IL-4Rαsv1, wherein a test preparation that binds to the IL-4Rαsv1 polypeptide, but does not bind to IL-4Rα isoform polypeptide that is not IL-4Rαsv1, contains one or more compounds that selectively bind to IL-4Rαsv1.

In another embodiment of the invention, a binding method is provided for screening for a compound able to bind selectively to an IL-4Rα isoform polypeptide that is not IL-4Rαsv1. The method comprises the steps: providing an IL-4Rαsv1 polypeptide comprising SEQ ID NO 5; providing a IL-4Rα isoform polypeptide that is not IL-4Rαsv1; contacting the IL-4Rαsv1 polypeptide and the IL-4Rα isoform polypeptide that is not IL-4Rαsv1 with a test preparation comprising one or more test compounds; and then determining the binding of the test preparation to the IL-4Rαsv1 polypeptide and the IL-4Rα isoform polypeptide that is not IL-4Rαsv1, wherein a test preparation that binds the IL-4Rα isoform polypeptide that is not IL-4Rαsv1, but does not bind IL-4Rαsv1, contains a compound that selectively binds the IL-4Rα isoform polypeptide that is not IL-4Rαsv1.

The above-described selective binding assays can also be performed with a polypeptide fragment of IL-4Rαsv1, wherein the polypeptide fragment comprises at least 10 consecutive amino acids that are coded by a nucleotide sequence that bridges the junction created by the splicing of the 3′ end of exon 10 to the 5′ end of intron 10A and the 3′ end of intron 10A to the 5′ end of exon 11. Similarly, the selective binding assays may also be performed using a polypeptide fragment of an IL-4Rα isoform polypeptide that is not IL-4Rαsv1, wherein the polypeptide fragment comprises at least 10 consecutive amino acids that are coded by: a) a nucleotide sequence that is contained within intron 10A of the IL-4Rα gene; or b) a nucleotide sequence that bridges the junction created by the splicing of 3′ end of exon 10 to the 5′ end of exon 11 of the IL-4Rα gene.

IL-4Rα Functional Assays

IL-4Rα encodes the alpha subunit of interleukin-4 receptor that plays an integral role in the cascade leading to the activation of STAT6 and the transcription of genes in response to allergic stimuli. IL-4Rα activity also depends on its phosphorylation state. The identification of IL-4Rαsv1 as a splice variant of IL-4Rα provides a means for screening for compounds that bind to IL-4Rαsv1 protein thereby altering the activity or regulation of IL-4Rαsv1. Assays involving a functional IL-4Rαsv1 polypeptide can be employed for different purposes, such as selecting for compounds active at IL-4Rαsv1; evaluating the ability of a compound to affect the signaling activity of each splice variant polypeptide; and mapping the activity of different IL-4Rαsv1 regions. IL-4Rαsv1 activity can be measured using different techniques such as: detecting a change in the intracellular conformation of IL-4Rαsv1; detecting a change in the intracellular location of IL-4Rαsv1; or by measuring the signaling activity of IL-4Rαsv1.

Recombinantly expressed IL-4Rαsv1 can be used to facilitate the determination of whether a compound is active at IL-4Rαsv1. For example, IL-4Rαsv1 can be expressed by an expression vector in a cell line and used in a co-culture growth assay, such as described in WO 99/59037, to identify compounds that bind to IL-4Rαsv1. For example, IL-4Rαsv1 can be expressed by an expression vector in a human kidney cell line 293 and used in a co-culture growth assay, such as described in U.S. patent application 20020061860, to identify compounds that bind to IL-4Rαsv1.

Several methods have been used to determine IL-4Rα activation or its function. Binding of IL-4 to its receptor induces expression of low affinity IgE receptor (F_{C}εR II or CD23) on B-lymphocytes via activity of STAT6 (Defrance et al., 1987 J. Exp. Med. 165:1459-67). Detection of surface expression of F_{C}εR II/CD23 on B-cells by FACS, using labeled anti-human CD23 monoclonal antibodies, is used as an assay for IL-4Rα responsiveness (see for example, So et al., 2002 FEBS Letters 518:53-50; Hershey et al., 1997 N. Engl. J. Med. 337:1720-1725; Perez-G et al., 2002 J. Immunol. 168:1428-1434; Ryan et al., 1996 Immunity 4:123-132). Electron mobility shift assays are used to determine the ability of IL-4Rα signaling to induce the DNA-binding activity of STAT6 toward α-³²P-labeled double-stranded probes consisting of a promoter STAT6 consensus site (Perez-G et al., 2002 J. Immunol. 168:1428-1434; Losman et al., 1999 J. Immunol. 162:3770-3774; Ryan et al., 1996 Immunity 4:123-132; Lu et al., 1997 J. Immunol. 159:1255-1264). Proliferation assays have also been used to determine IL-4Rα activity-induced B- or T-cell proliferation (Schnare et al., 1998 J. Immunol. 161:3484-3492; Reichel et al., 1997 J. Immunol. 158:5860-5867; Izuhara et al., 1995 Cell. Immunol. 163:254-259). IL-4 binding assays (Schulte et al., 1997 J. Exp. Med. 186:1419-1429) and measurement of IgE synthesis after IL-4 induced signaling (Mitsuyasu et al., 1999 J. Immunol. 162:1227-1231) have also been described to determine IL-4Rα activity and function. The transcriptional activity of STAT6 induced by IL-4Rα signaling can be determined by transfecting cells with a reporter construct containing the chloramphenicol acetyltransferase, luciferase, or other reporter gene under the control of STAT6 elements (see for example Losman et al., 1999 J. Immunol. 162:3770-3774; Mitsuyasu et al., 1999 J. Immunol. 162:1227-1231). Reporter gene activity can be measured by ELISA, thin layer chromatography, a luminometer, or a scintillation counter. A variety of other assays has been used to investigate the properties of IL-4Rα and therefore would also be applicable to the measurement of IL-4Rαsv1 function.

IL-4Rαsv1 functional assays can be performed using cells expressing IL-4Rαsv1 at a high level. These proteins will be contacted with individual compounds or preparations containing different compounds. A preparation containing different compounds where one or more compounds affect IL-4Rαsv1 in cells over-producing IL-4Rαsv1 as compared to control cells containing an expression vector lacking IL-4Rαsv1 coding sequences, can be divided into smaller groups of compounds to identify the compound(s) affecting IL-4Rαsv1 activity.

IL-4Rαsv1 functional assays can be performed using recombinantly produced IL-4Rαsv1 present in different environments. Such environments include, for example, cell extracts and purified cell extracts containing IL-4Rαsv1 expressed from recombinant nucleic acid; and the use of purified IL-4Rαsv1 produced by recombinant means that is introduced into a different environment suitable for measuring signaling activity.

The IL-4 receptor protein complex is a dimer, consisting of IL-4Rα and either γC or IL-13Rα1 subunits to form type I or type II receptors, respectively. Type I IL-4 receptors respond only to IL-4 ligand, while type II IL-4 receptors respond to both IL-4 and IL-13 (reviewed in Kelly-Welch et al., 2003 Science, 300:1527-1528). IL-4Rαsv1 functional assays can be performed using cells expressing either the type I or type II IL-4 receptor components such that IL-4 receptor dimerization occurs. Coding sequences for γC and IL-13Rα1 subunits have also been disclosed in Genbank, NM_{—}000206 and NM_{—}001560, respectively. Functional assays can be performed using cells producing IL-4Rαsv1/γC or IL-4Rαsv1/IL-13Rα1 receptor dimers. Such assays may be used to identify compounds that are active at either type I or type II IL-4 receptors, affect type I or type II receptor association, or affect type I or type II receptor signaling.

Modulating IL-4Rαsv1 Expression

IL-4Rαsv1 expression can be modulated as a means for increasing or decreasing IL-4Rαsv1 activity. Such modulation includes inhibiting the activity of nucleic acids encoding the IL-4Rα isoform target to reduce IL-4Rα isoform protein or polypeptide expression, or supplying IL-4Rα nucleic acids to increase the level of expression of the IL-4Rα target polypeptide thereby increasing IL-4Rα activity.

Inhibition of IL-4Rαsv1 Activity

IL-4Rαsv1 nucleic acid activity can be inhibited using nucleic acids recognizing IL-4Rαsv1 nucleic acid and affecting the ability of such nucleic acid to be transcribed or translated. Inhibition of IL-4Rαsv1 nucleic acid activity can be used, for example, in target validation studies.

A preferred target for inhibiting IL-4Rαsv1 is mRNA stability and translation. The ability of IL-4Rαsv1 mRNA to be translated into a protein can be effected by compounds such as anti-sense nucleic acid, RNA interference (RNAi) and enzymatic nucleic acid.

Anti-sense nucleic acid can hybridize to a region of a target mRNA. Depending on the structure of the anti-sense nucleic acid, anti-sense activity can be brought about by different mechanisms such as blocking the initiation of translation, preventing processing of mRNA, hybrid arrest, and degradation of mRNA by RNAse H activity.

RNA inhibition (RNAi) using shRNA or siRNA molecules can also be used to prevent protein expression of a target transcript. This method is based on the interfering properties of double-stranded RNA derived from the coding regions of the gene that disrupt the synthesis of protein from transcribed RNA.

Enzymatic nucleic acids can recognize and cleave other nucleic acid molecules. Preferred enzymatic nucleic acids are ribozymes.

General structures for anti-sense nucleic acids, RNAi and ribozymes, and methods of delivering such molecules, are well known in the art. Methods for using RNAi to modify IL-4Rα activity have been described previously (Ikizawa et al., 1995, Clin Exp Immunol. 100(3):383-9). Modified and unmodified nucleic acids can be used as anti-sense molecules, RNAi and ribozymes. Different types of modifications can affect certain anti-sense activities such as the ability to be cleaved by RNAse H, and can alter nucleic acid stability. Examples of references describing different anti-sense molecules, and ribozymes, and the use of such molecules, are provided in U.S. Pat. Nos. 5,849,902; 5,859,221; 5,852,188; and 5,616,459. Examples of organisms in which RNAi has been used to inhibit expression of a target gene include: C. elegans (Tabara, et al., 1999, Cell 99, 123-32; Fire, et al., 1998, Nature 391, 806-11), plants (Hamilton and Baulcombe, 1999, Science 286, 950-52), Drosophila (Hammond, et al., 2001, Science 293, 1146-50; Misquitta and Patterson, 1999, Proc. Nat. Acad. Sci. 96, 1451-56; Kennerdell and Carthew, 1998, Cell 95, 1017-26), and mammalian cells (Bernstein, et al., 2001, Nature 409, 363-6; Elbashir, et al., 2001, Nature 411, 494-8).

Increasing IL-4Rαsv1 Expression

Nucleic acids encoding IL-4Rαsv1 can be used, for example, to cause an increase in IL-4Rα activity or to create a test system (e.g., a transgenic animal) for screening for compounds affecting IL-4Rαsv1 expression. Nucleic acids can be introduced and expressed in cells present in different environments.

Guidelines for pharmaceutical administration in general are provided in, for example, Remington's Pharmaceutical Sciences, 18^{th} Edition, supra, and Modern Pharmaceutics, 2^{nd} Edition, supra. Nucleic acid can be introduced into cells present in different environments using in vitro, in vivo, or ex vivo techniques. Examples of techniques useful in gene therapy are illustrated in Gene Therapy & Molecular Biology: From Basic Mechanisms to Clinical Applications, Ed. Boulikas, Gene Therapy Press, 1998.

### EXAMPLES

Examples are provided below to further illustrate different features and advantages of the present invention. The examples also illustrate useful methodology for practicing the invention. These examples do not limit the claimed invention.

### Example 1

### Identification of IL-4Rαsv1 Using Microarrays

To identify variants of the “normal” splicing of exon regions encoding IL-4Rα, an exon junction microarray, comprising probes complementary to each splice junction resulting from splicing of the 11 exon coding sequences in IL-4Rα heteronuclear RNA (hnRNA), was hybridized to a mixture of labeled nucleic acid samples prepared from 44 different human tissue and cell line samples. Exon junction microarrays are described in Johnson et al. (2003 Science 302:2141-2144) and PCT patent applications WO 02/18646 and WO 02/16650. Materials and methods for preparing hybridization samples from purified RNA, hybridizing a microarray, detecting hybridization signals, and data analysis are described in Castle et al. (2003 Genome Biol. 4:R66.1-66.13), van't Veer, et al. (2002 Nature 415:530-536) and Hughes, et al. (2001 Nature Biotechnol. 19:342-7). Inspection of the exon junction microarray hybridization data (not shown) suggested that the structure of at least one exon junction of IL-4Rα mRNA was altered in some of the tissues examined, suggesting the presence of IL-4Rα splice variant mRNA populations. Reverse transcription and polymerase chain reactions (RT-PCR) were then performed using oligonucleotide primers complementary to exons 8 and 11 to confirm the exon junction array results and to allow the sequence structure of the splice variants to be determined.

### Example 2

### Confirmation of IL-4Rαsv1 Using RT-PCR

The structure of IL-4Rα mRNA in the region corresponding to exons 8 to 11 and was determined for a panel of human tissue and cell line samples using an RT-PCR based assay. PolyA purified mRNA isolated from 44 different human tissue and cell line samples was obtained from BD Biosciences Clontech (Palo Alto, Calif.), Biochain Institute, Inc. (Hayward, Calif.), and Ambion Inc. (Austin, Tex.). RT-PCR primers were selected that were complementary to sequences in exon 8 and exon 11 of the reference exon coding sequences in IL-4Rα (NM_{—}000418). Based upon the nucleotide sequence of IL-4Rα mRNA, the IL-4Rα exon 8 and exon 11 primer set (hereafter IL-4Rα₈₋₁₁ primer set) was expected to amplify a 318 base pair amplicon representing the “reference” IL-4Rα mRNA region. The IL-4Rα exon 8 forward primer has the sequence: 5′ GTCTGCCTGTTGTGCTATGTCAGCATC 3′ [SEQ ID NO 10]; and the IL-4Rα exon 11 reverse primer has the sequence: 5′ CCAGAGGACTGTCTTGCTGA TCTCCACT 3′ [SEQ ID NO 11].

Twenty-five ng of polyA mRNA from each tissue was subjected to a one-step reverse transcription-PCR amplification protocol using the Qiagen, Inc. (Valencia, Calif.), One-Step RT-PCR kit, using the following cycling conditions:

50° C. for 30 minutes;

95° C. for 15 minutes;

35 cycles of:
94° C. for 30 seconds;
63.5° C. for 40 seconds;
72° C. for 50 seconds; then
72° C. for 10 minutes.

RT-PCR amplification products (amplicons) were size fractionated on a 2% agarose gel. Selected amplicon fragments were manually extracted from the gel and purified with a Qiagen Gel Extraction Kit. Purified amplicon fragments were sequenced from each end (using the same primers used for RT-PCR) by Qiagen Genomics, Inc. (Bothell, Wash.).

At least two different RT-PCR amplicons were obtained from human mRNA samples using the IL-4R₈₋₁₁ primer set (data not shown). Every human tissue and cell line assayed exhibited the expected amplicon size of 318 base pairs for normally spliced IL-4Rα mRNA. However, in addition to the expected IL-4Rα amplicon of 318 base pairs, fetal lung, lung, lung carcinoma, thyroid, adrenal gland, thymus, and bone marrow also exhibited an amplicon of about 399 base pairs. The tissues in which IL-4Rαsv1 mRNA was detected are listed in Table 1:

TABLE 1
Tissue distribution of IL-4Rαsv1 polynucleotides

| Sample | IL-4Rαsv1 |
|---|---|
| Retina | |
| Pituitary | |
| Spinal Cord | |
| Brain, Cerebellum | |
| Brain, Frontal Lobe | |
| Brain, Medulla Oblongata | |
| Brain, Pons | |
| Brain, Putamen | |
| Brain, Thalamus | |
| Brain, Hippocampus | |
| Fetal Brain | |
| Fetal Kidney | |
| Fetal Liver | |
| Fetal Lung | X |
| Fetal Vertebra | |
| Heart | |
| Kidney | |
| Liver | |
| Pancreas | |
| Stomach | |
| Jejunum | |
| Ileum | |
| Colon, descending | |
| Colon tumor tissue | |
| Lung | X |
| Lung Carcinoma (A549) | X |
| Prostate | |
| Thyroid | X |
| Adipose | |
| Skin | |
| Skeletal Muscle | |
| Adrenal Gland | X |
| Thymus | X |
| Bone Marrow | X |
| Peripheral Leukocytes | |
| Uterus | |
| Placenta | |
| Ovary | |
| Testis | |
| Hela S3 | |
| Leukemia Promyelocytic (HL-60) | |
| Lymphoma Burkitt's (Raji) | |
| Melanoma (G361) | |
| Osteosarcoma (MG-63) | |

Sequence analysis of the about 399 base pair amplicon amplified using the IL-4Rα₈₋₁₁ primer set revealed that this amplicon form results from the retention of a portion of intron 10 (hereafter intron 10A [SEQ ID NO 3]) of the IL-4Rα hnRNA. That is, the longer form IL-4Rα amplicon is due to the insertion of intron 10A [SEQ ID NO 3] polynucleotide sequence. This splice variant form was designated IL-4Rαsv1 [SEQ ID NO 4]. Thus, the RT-PCR results confirmed the junction probe microarray data reported in Example 1 which suggested that IL-4Rα mRNA is composed of a mixed population of molecules wherein in at least one of the IL-4Rα mRNA splice junctions is altered.

### Example 3

### Cloning of IL-4Rαsv1

Microarray, RT-PCR, and sequencing data indicate that in addition to the normal IL-4Rα reference mRNA sequence, NM_{—}000418, encoding IL-4Rα protein, NP_{—}000409, a novel splice variant form of IL-4Rα mRNA also exist in many tissues.

Clones having a nucleotide sequence comprising the splice variant identified in Example 2 (hereafter referred to as IL-4Rαsv1) are isolated using a 5′ “forward” IL-4Rα primer and a 3′ “reverse” IL-4Rα primer, to amplify and clone the entire IL-4Rαsv1 mRNA coding sequences. The 5′ “forward” primer is designed for isolation of full length clones corresponding to the IL-4Rαsv1 splice variants and has the nucleotide sequence of 5′ ATGGGGTGGCTTTGCTCTG 3′ [SEQ ID NO 12]. The 3′ “reverse” primer is designed for isolation of full length clones corresponding to the IL-4Rαsv1 splice variant and has the nucleotide sequence of 5′ AGAGACCCTCATGTATGTGGGTC 3′ [SEQ ID NO 13].

RT-PCR

The IL-4Rαsv1 cDNA sequence is cloned using a combination of reverse transcription (RT) and polymerase chain reaction (PCR). More specifically, about 25 ng of lung polyA mRNA (BD Biosciences Clontech, Palo alto, Calif.) is reverse transcribed using Superscript II (Gibco/Invitrogen, Carlsbad, Calif.) and oligo d(T) primer (RESGEN/Invitrogen, Huntsville, Ala.) according to the Superscript II manufacturer's instructions. For PCR, 1 μl of the completed RT reaction is added to 40 μl of water, 5 μl of 10× buffer, 1 μl of dNTPs and 1 μl of enzyme from the Clontech (Palo Alto, Calif.) Advantage 2 PCR kit. PCR is done in a Gene Amp PCR System 9700 (Applied Biosystems, Foster City, Calif.) using the IL-4R “forward” and “reverse” primers. After an initial 94° C. denaturation of 1 minute, 35 cycles of amplification are performed using a 30 second denaturation at 94° C. followed by a 40 second annealing at 63.5° C. and a 50 second synthesis at 72° C. The 35 cycles of PCR are followed by a 10 minute extension at 72° C. The 50 μl reaction is then chilled to 4° C. 10 μl of the resulting reaction product is run on a 1% agarose (Invitrogen, Ultra pure) gel stained with 0.3 μg/ml ethidium bromide (Fisher Biotech, Fair Lawn, N.J.). Nucleic acid bands in the gel are visualized and photographed on a UV light box to determine if the PCR has yielded products of the expected size, in the case of the predicted IL-4Rsv1 mRNA, a product of about 2556 base pairs. The remainder of the 50 μl PCR reactions from lung is purified using the QIAquik Gel extraction Kit (Qiagen, Valencia, Calif.) following the QIAquik PCR Purification Protocol provided with the kit. About 50 μl of product obtained from the purification protocol is concentrated to about 6 μl by drying in a Speed Vac Plus (SC110A, from Savant, Holbrook, N.Y.) attached to a Universal Vacuum System 400 (also from Savant) for about 30 minutes on medium heat.

Cloning of RT-PCR Products

About 4 μl of the 6 μl of purified IL-4Rαsv1 RT-PCR product from lung are used in a cloning reaction using the reagents and instructions provided with the TOPO TA cloning kit (Invitrogen, Carlsbad, Calif.). About 2 μl of the cloning reaction is used following the manufacturer's instructions to transform TOP10 chemically competent E. coli provided with the cloning kit. After the 1 hour recovery of the cells in SOC medium (provided with the TOPO TA cloning kit), 200 μl of the mixture is plated on LB medium plates (Sambrook, et al., in Molecular Cloning, A Laboratory Manual, 2^{nd} Edition, Cold Spring Harbor Laboratory Press, 1989) containing 100 μg/ml Ampicillin (Sigma, St. Louis, Mo.) and 80 μg/ml X-GAL (5-Bromo-4-chloro-3-indoyl B-D-galactoside, Sigma, St. Louis, Mo.). Plates are incubated overnight at 37° C. White colonies are picked from the plates into 2 ml of 2× LB medium. These liquid cultures are incubated overnight on a roller at 37° C. Plasmid DNA is extracted from these cultures using the Qiagen (Valencia, Calif.) Qiaquik Spin Miniprep kit. Twelve putative IL-4Rαsv1 clones are identified and prepared for a PCR reaction to confirm the presence of the expected IL-4Rαsv1 exon 10 to intron 10A and intron 10A to exon 11 splice variant structures. A 25 μl PCR reaction is performed as described above (RT-PCR section) to detect the presence of IL-4Rαsv1, except that the reaction includes miniprep DNA from the TOPO TA/IL-4Rαsv1 ligation as a template. About 10 μl of each 25 μl PCR reaction is run on a 1% agarose gel and the DNA bands generated by the PCR reaction are visualized and photographed on a UV light box to determine which minipreps samples have PCR product of the size predicted for the corresponding IL-4Rαsv1 splice variant mRNA. Clones having the IL-4Rαsv1 structure are identified based upon amplification of an amplicon band of 399 base pairs, whereas a normal reference IL-4Rα clone will give rise to an amplicon band of 318 base pairs. DNA sequence analysis of the IL-4Rαsv1 cloned DNAs confirms a polynucleotide sequence representing the retention of intron 10A.

The polynucleotide sequence of IL-4Rαsv1 mRNA (SEQ ID NO 4) contains an open reading frame that encodes an IL-4Rαsv1 protein (SEQ ID NO 5) similar to the reference IL-4Rα protein (NP_{—}000409), but retaining amino acids encoded by a 81 base pair region corresponding to a portion of intron 10 of the full length coding sequence of the reference IL-4Rα mRNA (NM_{—}000418). The insertion of the 81 base pair region does not change the protein translation reading frame in comparison to the reference IL-4Rα protein reading frame. Therefore, the IL-4Rαsv1 protein has an additional internal 27 amino acid region as compared to the reference IL-4Rα (NP_{—}000409).

All patents, patent publications, and other published references mentioned herein are hereby incorporated by reference in their entireties as if each had been individually and specifically incorporated by reference herein. While preferred illustrative embodiments of the present invention are shown and described, one skilled in the art will appreciate that the present invention can be practiced by other than the described embodiments, which are presented for purposes of illustration only and not by way of limitation. Various modifications may be made to the embodiments described herein without departing from the spirit and scope of the present invention. The present invention is limited only by the claims that follow.

1. A purified human nucleic acid comprising SEQ ID NO 4, or the complement thereof.

2. The purified nucleic acid of claim 1, wherein said nucleic acid comprises a region encoding SEQ ID NO 5.

3. The purified nucleic acid of claim 1, wherein said nucleotide sequence encodes a polypeptide consisting of SEQ ID NO 5.

4. A purified polypeptide comprising SEQ ID NO 5.

5. The polypeptide of claim 4, wherein said polypeptide consists of SEQ ID NO 5.

6. A method for screening for a compound able to bind to IL-4Rαsv1 comprising the steps of:
(a) expressing a polypeptide comprising SEQ ID NO 5 from recombinant nucleic acid;
(b) providing to said polypeptide a test preparation comprising one or more test compounds; and
(c) measuring the ability of said test preparation to bind to said polypeptide.

7. The method of claim 6, wherein said steps (b) and (c) are performed in vitro.

8. The method of claim 6, wherein said steps (a), (b), and (c) are performed using a whole cell.

9. The method of claim 6, wherein said polypeptide is expressed from an expression vector comprising a polynucleotide encoding SEQ ID NO 5.

10. A method of screening for compounds able to bind selectively to IL-4Rαsv1 comprising the steps of:
(a) providing a IL-4Rαsv1 polypeptide comprising SEQ ID NO 5;
(b) providing one or more IL-4Rα isoform polypeptides that are not IL-4Rαsv1;
(c) contacting said IL-4Rαsv1 polypeptide and said IL-4Rα isoform polypeptide that is not IL-4Rαsv1 with a test preparation comprising one or more compounds; and
(d) determining the binding of said test preparation to said IL-4Rαsv1 polypeptide and to said IL-4Rα isoform polypeptide that is not IL-4Rαsv1, wherein a test preparation that binds to said IL-4Rαsv1 polypeptide, but does not bind to said IL-4Rα polypeptide that is not IL-4Rαsv1, contains a compound that selectively binds said IL-4Rαsv1 polypeptide.

11. The method of claim 10, wherein said IL-4Rαsv1 polypeptide is obtained by expression of said polypeptide from an expression vector comprising a polynucleotide encoding SEQ ID NO 5.

12. The method of claim 11, wherein said polypeptide consists of SEQ ID NO 5.

13. A method for screening for a compound able to bind to or interact with a IL-4Rαsv1 protein or a fragment thereof comprising the steps of:
(a) expressing a IL-4Rαsv1 polypeptide comprising SEQ ID NO 5 or fragment thereof from a recombinant nucleic acid;
(b) providing to said polypeptide a labeled IL-4Rα ligand that binds to said polypeptide and a test preparation comprising one or more compounds; and
(c) measuring the effect of said test preparation on binding of said labeled IL-4Rα ligand to said polypeptide, wherein a test preparation that alters the binding of said labeled IL-4Rα ligand to said polypeptide contains a compound that binds to or interacts with said polypeptide.

14. The method of claim 13, wherein said steps (b) and (c) are performed in vitro.

15. The method of claim 13, wherein said steps (a), (b) and (c) are performed using a whole cell.

16. The method of claim 13, wherein said polypeptide is expressed from an expression vector.

17. The method of claim 13, wherein said IL-4Rαsv1 ligand is an IL-4Rα inhibitor.

18. The method of claim 16, wherein said expression vector comprises SEQ ID NO 4 or a fragment of SEQ ID NO 4.

19. The method of claim 16, wherein said polypeptide comprises SEQ ID NO 5 or a fragment of SEQ ID NO 5.

20. A method of screening for IL-4Rαsv1 activity comprising the steps of:
(a) contacting a cell expressing a recombinant nucleic acid encoding IL-4Rαsv1 comprising SEQ ID NO 5 with a test preparation comprising one or more test compounds; and
(b) measuring the effect of said test preparation on IL-4Rα signalling.
